(19) United States
(12) Patent Application Publication
(10) Pub. No.: US 2021/0393908 A1
(43) Pub. Date: Dec. 23, 2021

(54) Oral Suction Device with Anti-Infective Protection

(71) Applicant: Nina McLain, Philadelphia, MS (US)

(72) Inventor: Nina McLain, Philadelphia, MS (US)

(21) Appl. No.: 17/350,847

(22) Filed: Jun. 17, 2021

Related U.S. Application Data
(60) Provisional application No. 63/040,346, filed on Jun. 17, 2020.

(51) Int. Cl. A61M 16/04; A61L 29/08; A61L 29/14; A61L 29/16; A61L 29/04

(52) U.S. Cl. CPC A61M 16/0463; A61M 16/0497; A61L 29/085; A61L 29/145; A61L 29/146; A61L 2300/21; A61L 29/04; A61M 2205/32; A61M 2205/0238; A61L 2300/404; A61L 29/16

(57) ABSTRACT

The improvement device provides an oral suction system for use with endo-tracheal tubes. The device provides alternative anti-infective components and a removable and/or disposable external sleeve, both for mitigation or elimination of risks for viral and/or bacterial contamination of the patient and healthcare professional.

### CROSS-REFERENCE TO RELATED APPLICATION

This application claims priority from U.S. Provisional Patent Application Ser. No. 63/040,346 filed Jun. 17, 2020. The entirety of the provisional application is incorporated herein by reference.

### BRIEF DESCRIPTION OF THE DRAWINGS

In the accompanying drawings:

FIG. 1 shows the device overview, elements, and interconnections.

FIG. 2 shows the device and the integrated sleeve of the device covering the device, specifically the covered perforated sponge, upon removal from the patient.

### DESCRIPTION OF EMBODIMENTS

The enhanced safety oral suction device improves upon known oral suction devices. The system protects the patient and healthcare professionals and minimizes the risks of infection, for example, due to aspirations and sputum overflow, by utilizing antiviral and/or anti-infective components and, in an alternative embodiment, an integrated plastic sleeve. Examples of anti-infective components that may be used with the system include zinc picolinate, antibacterials and/or antiseptics, or a combination thereof, for example. The integrated plastic sleeve, or other similar flexible material covering, may be disposable for post-use removal. The sleeve may preferentially be clear to aid in viewing inside the sleeve. The improved oral suction device can be used with other systems and devices such as endo-tracheal tubes.

The improved oral suction device disclosed is for a patient in need thereof and is comprised of a coated sponge, for example a hydrogel-coated or non-hydrogel-coated sponge, wherein the sponge may be a foam-type sponge and may also be perforated; a length of flexible tube, wherein the tube length and tube diameter are variable based on factors such as specific patient needs, and wherein the tube is preferably flexible and clear to enhance visibility into the tube; an endo-tracheal tube clip for attaching to an endo-tracheal tube; a wall suction tubing connector or connecting means; and a cap or connection cap for the wall suction tubing connector. Further, in an alternative embodiment, an integrated, preferably plastic, protective flexible cover sleeve is included and can be pulled down over the oral suction device as it is removed from the patient, or preferably kept static and the sponge and a portion of the flexible tube manually pulled into it, to prevent sputum from splashing. Moreover, the system and device and the integrated, preferably clear plastic, sleeve are disposable. The sleeve may be comprised of paper, plastic, or a combination thereof. Moreover, the device may be alternatively pre-lubricated. Additionally, depth placement markings and/or radiopaque markings for system placement and use with a patient are or can be included on the flexible tube of the system and device to aid in placement and to monitor its use. Certain features of the improvement include the depth and/or radiopaque markings, the endo-tracheal tube clip, and the plastic removable sleeve.

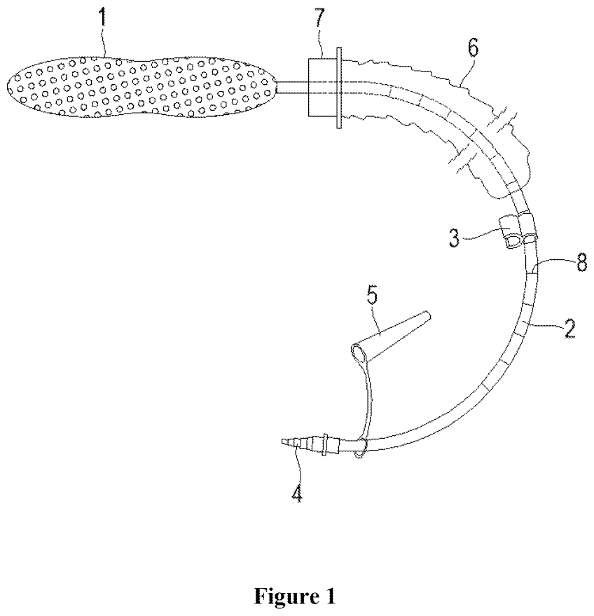

FIG. 1 shows a pictorial representation of the system and device overview, device elements, and system interconnections. Sponge 1 is connected and fixed to a first end of tube 2, wherein tube 2 is preferentially flexible plastic or other similar flexible material and may be clear. Sponge 1 may be perforated and may include hydrogel or non-hydrogel coating, or a combination thereof. Endo-tracheal tube clip 3 is preferably plastic, is formed in a c-clip fashion, and is slidably connected to tube 2 and adjustably movable along the usable length of tube 2. The opposite and second end of tube 2 is connected and fixed to connector 4, which provides a connection to wall suction tubing, for example. Connection cap 5 is slidably connected to tube 2 and located typically near connector 4 and utilized to cover connector 4. One end of plastic sleeve 6 is connected and fixed to or near one circular end of sleeve end cap 7 and the other circular end of sleeve end cap 7 is open to accept sponge 1. The plastic sleeve-end of sleeve end cap 7 has an extended raised circular lip that is larger in diameter than the sleeve end cap 7 and connected to plastic sleeve 6. The plastic sleeve 6 is fixed on or near the sleeve end cap 7 to aid in sponge 1 removal. The other end of plastic sleeve 6 is sufficiently open only to accept tube 2. Markings 8 are located on tube 2 and can be physical marks, radiopaque marks, or a combination thereof. Tube clip 3 may also be located close in proximity to connection cap 5. Further, integrated plastic sleeve 6 is optional and is preferably comprised of clear plastic material for viewing contents therein. Plastic sleeve 6 may be attached near the endo-tracheal tube clip 3 down to and toward sponge 1. The coated and/or perforated sponge 1 facilitates the flow of oral secretions. The integrated clear plastic sleeve 6 can be pulled down over the device as sponge 1 and a portion of tube 2 is removed from the patient, or alternatively, sleeve 6 can remain and be held stationary by the user as tube 2 and sponge 1 are drawn in or pulled into plastic sleeve 6 for disposal.

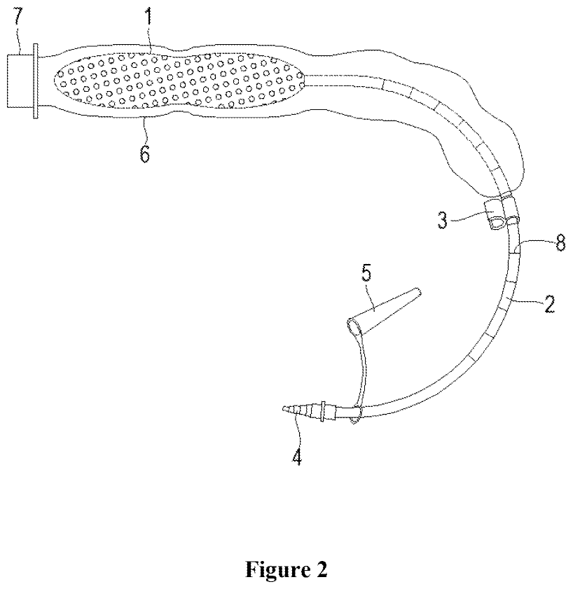

FIG. 2 shows a pictorial representation of the system and device, with the integrated plastic sleeve 6 over and covering the device, specifically the coated and/or coated perforated sponge 1 and a portion of tube 2, typically upon removal from the patient. A portion of tube 2 and all of sponge 1 are typically pulled in to sleeve 6 by a user holding the extended raised circular lip of sleeve end cap 7 stationary in place and pulling the portion of tube 2 and all of sponge 1 into the sleeve 6 for disposal.

The oral suction device can be used in conjunction with an endo-tracheal tube. The device helps eliminate or minimize risks of hospital-acquired infections due to aspirations and sputum overflow, for example. The device can be used for patients who are stable and on mechanical ventilation and particularly for patients on long-term care and support, who are typically at higher risk for aspiration and complications from sputum overflow.

The pre-lubrication solution of the device is optional and aids in the insertion into the throat so that the device may alternatively be pre-lubricated with at least one such solution. The device can be formed to fit the anatomy of the human or animal throat without creating hotspots or irritation. Moreover, the system allows for improved patient care and ease of use and for protection of healthcare providers and provides for elimination or reduction of respiratory infections and contamination.

The device provides improvements over known medical oral suction devices including the use of a hydrogel or non-hydrogel coating that can be impregnated with anti-infective components to provide protection for the patient and caregiver. These additive components include, for example, antiviral additive zinc picolinate, antibacterial additives, and/or antiseptic additives, or a combination thereof, to mitigate or eliminate viral and/or bacterial contamination or transfer. The device includes an external covering over the oral suction device, that accepts and contains the sponge when removed from a patient, utilizing a plastic or paper sleeve, or a combination thereof, or similar flexible material sleeve, that is disposable for post-use removal and protection of both patient and health professional(s) and for mitigation and prevention of possible contamination due to sputum and/or secretions that may occur during and after device use. A patient can be any animal, including human.

The oral suction device is an indwelling sponge system with a suction catheter and a hydrogel or non-hydrogel coating used to intermittently or continuously suction secretions from the oral cavity, the hypopharynx, and/or the supraglotic regions of a patient. The oral suction device on which this disclosure provides an improvement is disclosed in U.S. Pat. No. 10,099,027. That technology and disclosure includes that the gel of that device could be impregnated with antibiotics. The improvements disclosed herein comprise antiviral, antibacterial and/or antiseptic components to the coated sponge for patient and health professional protection. Further is disclosed removable and/or disposable sleeve protection for mitigation or elimination of contamination due to patient sputum and secretions. Impregnating the hydrogel or non-hydrogel sponge with known antiviral non-pharma and pharma components, for example, can reduce and/or eliminate contamination and potential spread of disease. Further, the device can provide one treatment toward COVID-19 and other respiratory viruses carried in droplet form.

The device provides a semi-closed suction system and can be used to protect individuals and/or animals from infections. It can reduce or eliminate contamination by highly contagious diseases. The device disclosed herein provides improvements of and is based upon a known oral suction device, such improvements comprising anti-infective capabilities and removable and disposable sleeve protection for secretion removal to protect both patient and healthcare providers. The disclosure of U.S. Pat. No. 10,099,027 is incorporated herein by reference.

The improved oral suction device disclosed herein comprises a suction catheter having a suction portion at a first end, a shell surrounding the suction portion, and a suction tubing connector on a second end opposite the first end, wherein the shell comprises a hydrogel or non-hydrogel coating, the suction portion of the suction catheter comprises a plurality of holes, the shell comprises a plurality of holes, and the plurality of holes in the shell are configured, in use, to allow removal therethrough of fluids from an oral cavity of a patient. The oral suction device further comprises a sponge connected to and surrounding the suction portion, wherein the shell surrounds the sponge, and wherein the improvement comprises an anti-infective additive impregnated within the coated sponge for mitigating or eliminating viral and/or bacterial contamination or transfer, and a removable, disposable, and/or flexible external sleeve covering the sponge and a portion of the flexible tube of the improved oral suction device, upon removal of the sponge from the mouth, for post-use removal and protection of the patient and health professional and for mitigation and/or prevention of any possible contamination due to sputum and/or secretions occurring both during and after use of the improved oral suction device. The flexible sleeve is fixed to or near one circular end of a sleeve end cap with an extended raised circular lip on the tube end of the sleeve end cap for ease of drawing the sponge into the sleeve for disposal.

The improved device utilizes anti-infective additives including antiviral additive zinc picolinate, an antibacterial, an antiseptic, or a combination thereof. The external flexible sleeve can be plastic, paper, or a combination thereof.

An alternative embodiment of the device consists of an improved suction device that comprises a suction catheter having a suction portion, a sponge connected to and surrounding the suction portion, a shell comprising a hydrogel or non-hydrogel coating, surrounding the sponge and the suction portion, a suction tubing connector on a first end of the suction catheter, an electronic temperature probe on a second end of the suction catheter opposite the suction tubing connector, an esophageal stethoscope comprising a stethoscope tube having a listening end, wherein the stethoscope tube enters the suction catheter near the suction tubing connector and is located within the suction catheter, a seal separating the listening end of the esophageal stethoscope from the suction portion, a stethoscope connector on a first end of the stethoscope tube opposite the listening end, and one or more leads in electrical communication with the electronic temperature probe.

The device improvement comprises an antiviral, antibacterial and/or antiseptic additive impregnated within the hydrogel or non-hydrogel coated sponge for mitigating or eliminating viral and/or bacterial contamination or transfer and a removable, disposable, and flexible external sleeve covering the improved oral suction device for post-use removal and protection of the patient and health professionals and for mitigation and/or prevention of possible contamination due to sputum and/or secretions that occur during and after use of the improved oral suction device. The device of this embodiment utilizes the antiviral additive zinc picolinate, an antibacterial, an antiseptic, or a combination thereof.

Further, the improvement device alternative embodiment uses an external flexible sleeve comprised of paper, plastic, or a combination thereof. The external plastic flexible sleeve can be composed of polyvinyl chloride (PVC), polyurethane, silicone, or a combination thereof.

The hydrogel coating of the improved device is at least one member selected from the group consisting of polyacrylamide; agar-agar; polyvinyl pyrrolidone; silicone hydrogels; polymers and copolymers of 2-hydroxyethylmethacrylate, glyceryl methacrylate, methyl methacrylate, N-vinyl pyrrolidone, N-vinyl-2-pyrrolidone, 2-methacryloyloxyethyl phosphorylcholine, ethoxyethylmethacrylate and methacrylic acid. Further, the hydrogel may also comprise thermoplastic polyurethane and water. Still further, the hydrogel may also comprise polyvinyl pyrrolidone, polyethylene glycol, and agar-agar and water.

The non-hydrogel coating of the improved suction device may be comprised of any medical grade silicone material, medical grade thermoplastic elastomers, or any approved medical-grade polymer such as poly(dimethyl siloxane), trimethylsilyloxy end-blocked polydimethylsiloxanes, polyurethane, acetal copolymer, acetal homopolymer, polyethylene teraphthalate polyester, polytetrafluoroethylene, ethylene-chlorotrifluoro-ethylene, polybutylene terephthalate-polyester, polyvi-nylidene fluoride, polyphenylene oxide, polyetheretherke-tone, polycarbonate, polyethylenes, polypropylene homopolymer, polyphenylsulfone, polysulfone, polyether-sulfone, and polyarylethersulfone.

Another alternative embodiment consists of a device comprising a suction catheter having a suction portion at a first end, a sponge connected to the suction portion, a hydrogel or non-hydrogel coating on the sponge, and a suction tubing connector on a second end of the suction catheter opposite the first end of the catheter.

The improved system and device can be packaged within a thermoform PTEG tray, for example, and a lid or cover that seals to the tray and provides a safety seal onto the tray for easy removal when the device is ready to be removed and used. The tray may be molded to form or fit the device and its elements.

The improved oral suction system may be used in conjunction with the use of one or more of a feeding tube, an endotracheal tube, and/or a central line (intra-jugular, clavicular, or super clavicular, for example). The hydrogel and/or non-hydrogel coating of the device provides for ease of insertion and removal into and from the throat, so that little or no force is required to insert or remove the device. The device may also be pre-lubricated for ease of application. Additionally, the device may be efficiently placed in a patient's mouth when the device markings align with the patient incisors (human), for example, or by using other measurement criteria. Once placed, the device can be secured to a vent, for example by a “c”-type clip to a vent tube, and connected to a suction or wall suction device.

The improved device can be removed from the patient and replaced routinely, depending on patient factors and caregiver facility policies. The device is disposable and can be removed from the suction or wall suction device and the integrated, preferably plastic, sleeve utilized to minimize possible patient fluid splash. The device can be bundled with the suction line and wrapped or contained in a watertight bag or container to contain all sputum and secretions, and ultimately disposed in a biohazard container.

All parameters presented herein including, but not limited to, temperatures, pressures, volumes, dimensions, times, sizes, amounts, distances, quantities, ratios, weights, percentages, and/or similar features and data and the like, for example, represent approximate values and can vary with the possible embodiments described and those not necessarily described but encompassed by the invention. Recitations of numerical values and/or numerical ranges can be read to include the term ‘about’. Unless defined otherwise, all technical and scientific terms used herein have the same meaning as commonly understood to one of ordinary skill in the art to which the invention belongs. Further, references to “a”, “an”, and “the” concerning any particular item, component, material, or product include plural references and are defined as at least one and could be more than one, unless the context clearly dictates otherwise. The terminology employed is for the purpose of describing particular embodiments and is not intended to be limiting in any way.

While specific embodiments have been described in detail, those with ordinary skill in the art will appreciate that various modifications and alternatives to those details could be developed in light of the overall teachings of the disclosure. Accordingly, the particular arrangements disclosed are meant to be illustrative only and not limiting as to the scope of the invention, which is to be given the full breadth of the appended claims or embodiments and any and all equivalents thereof.

What is claimed is:

1. An improved oral suction device for a patient in need thereof for use in conjunction with an endo-tracheal tube and to minimize the risk of infections, the device comprising a coated sponge, a flexible tube, an endo-tracheal tube clip, a wall suction tubing connector or connecting means, a connection cap, and an optional pre-lubricating solution.

2. The device of claim 1, further comprising a removable, integrated, plastic sleeve into which can be pulled the coated sponge and a portion of the flexible tube as the coated sponge and portion of the flexible tube are removed from the patient to minimize sputum splash, a sleeve end cap with an extended raised circular lip larger in diameter than the sleeve end cap on the sleeve end cap and connected to the plastic sleeve, and depth placement markings on the flexible tube to aid in device placement and monitoring, wherein the plastic sleeve is fixed on or near the sleeve end cap to aid in sponge removal and wherein the depth placement markings are physical marks, radiopaque marks, or a combination thereof.

3. An improved oral suction device for a patient in need thereof comprising a suction catheter having a suction portion at a first end, a shell surrounding the suction portion, and a suction tubing connector on a second end opposite the first end, wherein the shell comprises a hydrogel or non-hydrogel coating, the suction portion of the suction catheter comprises a plurality of holes, the shell comprises a plurality of holes, and the plurality of holes in the shell are configured to, in use, allow removal therethrough of fluid from an oral cavity of a patient, the oral suction device further comprising a sponge surrounding the suction portion, wherein the shell surrounds the sponge, and wherein the improvement comprises:
an anti-infective additive impregnated within the coated sponge for mitigating or eliminating viral and/or bacterial contamination or transfer; and
a removable, disposable, and/or flexible external sleeve covering the improved oral suction device for post-use removal and protection of the patient and health professional and for mitigation and/or prevention of possible contamination due to sputum and/or secretions that occur during and after use of the improved oral suction device.

4. The improved oral suction device of claim 3, wherein the anti-infective additive is antiviral additive zinc picolinate, an antibacterial, an antiseptic, or a combination thereof.

5. The improved oral suction device of claim 3, wherein the external sleeve is paper, plastic, or a combination thereof.

6. An improved oral suction device for a patient in need thereof comprising a suction catheter having a suction portion, a sponge surrounding the suction portion, wherein the sponge is radiopaque, a shell comprising a hydrogel or non-hydrogel coating, surrounding the sponge and the suction portion, a suction tubing connector on a first end of the suction catheter, an electronic temperature probe on a second end of the suction catheter opposite the suction tubing connector, an esophageal stethoscope comprising a stethoscope tube having a listening end, wherein the stethoscope tube enters the suction catheter near the suction tubing connector and is located within the suction catheter, a seal separating the listening end of the esophageal stethoscope from the suction portion, a stethoscope connector on a first end of the stethoscope tube opposite the listening end, and one or more leads in electrical communication with the electronic temperature probe, and wherein the improvement comprises:
an anti-infective additive impregnated within the coated sponge for mitigating or eliminating viral and/or bacterial contamination or transfer; and
a removable, disposable, and/or flexible external sleeve covering the improved oral suction device for post-use removal and protection of the patient and health professional and for mitigation and/or prevention of possible contamination due to sputum and/or secretions that occurs during and after use of the improved oral suction device.

7. The improved oral suction device of claim 6, wherein the anti-infective additive is zinc picolinate, an antibacterial additive, an antiseptic additive, or a combination thereof.

8. The improved oral suction device of claim 6, wherein the external sleeve is paper, plastic, or a combination thereof.

9. The improved oral suction device of claim 8, wherein the external sleeve is plastic and is comprised of polyvinyl chloride, polyurethane, silicone, or a combination thereof.

10. The improved oral suction device of claim 3, wherein the hydrogel coating is at least one member selected from the group consisting of polyacrylamide; agar-agar; polyvinyl pyrrolidone; silicone hydrogels; polymers and copolymers of 2-hydroxyethylmethacrylate, glyceryl methacrylate, methyl methacrylate, N-vinyl pyrrolidone, N-vinyl-2-pyrrolidone, 2-methacryloyloxyethyl phosphorylcholine, ethoxyethylmethacrylate and methacrylic acid; thermoplastic polyurethane and water; and polyvinyl pyrrolidone, polyethylene glycol, and agar-agar and water.

11. The improved oral suction device of claim 3, wherein the non-hydrogel coating is a medical grade polymer.

12. The improved oral suction device of claim 11, wherein the medical grade polymer is any silicone material, thermoplastic elastomers, or any approved polymer consisting of poly(dimethyl siloxane), trimethylsilyloxy end-blocked polydimethylsiloxanes, polyurethane, acetal copolymer, acetal homopolymer, polyethylene teraphthalate polyester, polytetrafluoroethylene, ethylene-chlorotrifluoro-ethylene, polybutylene terephthalate-polyester, polyvi-nylidene fluoride, polyphenylene oxide, polyetheretherke-tone, polycarbonate, polyethylenes, polypropylene homopolymer, polyphenylsulfone, polysulfone, polyether-sulfone, and polyarylethersulfone.
